(19) United States
(12) Patent Application Publication
(10) Pub. No.: US 2024/0410878 A1
(43) Pub. Date: Dec. 12, 2024

(54) EX VIVO HUMAN MODEL INTENDED FOR EVALUATING THE VACCINE POTENTIAL OF A COMPOSITION

(71) Applicant: GENOSKIN, Toulouse (FR)

(72) Inventors: Manon SCHOLAERT, Toulouse (FR); Nicolas GAUDENZIO, Tournefeuille (FR); Emeline PAGES, Ramonville Saint Agne (FR)

(21) Appl. No.: 18/785,910

(22) Filed: Jul. 26, 2024

Related U.S. Application Data
(63) Continuation-in-part of application No. PCT/EP2023/052096, filed on Jan. 27, 2023.

(30) Foreign Application Priority Data
Jan. 27, 2022 (FR) FR2200690

(51) Int. Cl. G01N 33/50

(52) U.S. Cl. CPC G01N 33/5082

(57) ABSTRACT

The present invention relates to an in vitro method intended to determine the vaccine potential of a composition comprising the steps of: ia) transcutaneous administration of the composition to a skin explant, comprising the epidermis, dermis and skin appendages as well as a thickness of at least 5 millimeters of hypodermis; ib) determination of the activation status of antigen-presenting cells within the skin explant; and ii) determination of the vaccine potential of the composition.

### CROSS REFERENCE TO RELATED APPLICATIONS

This application is a Continuation-in-Part of PCT International Application No. PCT/EP2023/052096, filed on Jan. 27, 2023, which claims priority under 35 U.S.C. 119(a) to Patent Application No. FR2200690, filed in France on Jan. 27, 2022, all of which are hereby expressly incorporated by reference into the present application.

### FIELD OF THE INVENTION

The present invention relates to the field of vaccination and proposes, more specifically, the first ex vivo human model that makes it possible to determine the vaccine potential of a composition.

### PRIOR ART

To ensure its protection, the human body possesses two types of defense mechanisms, which are innate immunity and adaptive immunity.

Innate immunity allows the body to immediately defend itself against infectious agents. Conversely, adaptive (or acquired) immunity confers later, but more lasting, protection to the body.

Regarding innate immunity, it is immediate in the event of attack by an infectious agent. Now, this is an antigen-independent response to said infectious agent. In addition, this immune response is of comparable intensity with each exposure to the same infectious agent.

Adaptive immunity requires a learning phase for the body. Following the interaction between an infectious agent and innate immunity, adaptive immunity springs into action in lymphoid tissues, especially in the lymph nodes and the spleen. Several mechanisms then come into play:

1—The antigen (infectious agent) directly activates B cells, which have specific receptors. The activated B cells then become plasma cells, which will secrete specific antibodies for the destruction of the antigen (humoral immunity).

2—The antigen (infectious agent) is presented to T cells by antigen-presenting cells (e.g. dendritic cells). Antigen-presenting cells activate T cells, which differentiate into:
Cytotoxic T cells (CD8+), which destroy infected cells (cellular immunity);
Helper T cells (CD4+), or T follicular helper cells, which stimulate B cells to produce a greater quantity of antibodies and memory cells, which will then lodge in the bone marrow.

Adaptive immunity therefore requires, after a first contact with an infectious agent, at least 2 to 3 weeks to be established. This immunity is dependent on and specific for the antigens of an infectious agent. The associated immune response increases in intensity following each contact with the same infectious agent.

The vaccine strategy aims to develop adaptive immunity using antigens from a specific infectious agent. Currently, obtaining a composition with good vaccine potential is a complex exercise. Indeed, primarily, it is important that said composition enables the proper activation of the antigen-presenting cells, so as to be able to induce a specific immune response. Simultaneously, it is preferable that this composition does not induce too much of an inflammatory response (i.e. limited degranulation of mast cells).

Today, the only option for estimating the vaccine potential of a composition lies in animal models which still leave many unknowns about the real vaccine potential of a given composition in humans.

Therefore, there is a need for a tool that would make it possible to easily and quickly determine, without resorting to animals, the vaccine potential in humans of an injectable composition.

### SUMMARY OF THE INVENTION

The inventors previously developed an ex vivo model of human skin that makes it possible to test the subcutaneous injection of a solution without needing an animal.

The inventors had also demonstrated that not only were mast cells stably present in said human skin model, but also that they remained functional with their degranulation capacity intact.

Furthermore, the inventors have demonstrated that the antigen-presenting cells are also present in said skin model and moreover that they have an intact maturation capacity. This result was all the more unexpected since, following the cultivation of human skin, these presenting cells were described as migrating out of the skin explant to the culture medium. (LENZ et al., J. Clin. Invest., vol.92, p: 2587-2596, 1993; POPE et al., J. Invest. Dermatol., vol.104(1), p: 11-17, 1995; RATZINGER et al., J. Immunol., vol.168, p: 4361-4371; 2002; KIVINEN et al., Experimental Dermatology, vol.12, p: 53-60, 2003).

In light of this discovery, the inventors had the idea of orienting this model so as to determine the vaccine potential of a composition by monitoring the response of these antigen-presenting cells following the injection of said composition.

The inventors have finally demonstrated that this new model effectively makes it possible to determine the vaccine potential of a composition ex vivo, which constitutes a major achievement in this field of technology.

Thus, an initial object of the invention relates to an in vitro method intended for determining the vaccine potential of a composition comprising the steps of:
ia) transcutaneous administration of the composition to a skin explant comprising the epidermis, the dermis and the skin appendages as well as a thickness of at least 5 millimeters of hypodermis;
ib) determination of the activation status of antigen-presenting cells within the skin explant; and
ii) determination of the vaccine potential of the composition.

According to a preferred embodiment, the method further comprises a step ic) of determining the possible migration of the antigen-presenting cells within the skin explant.

According to another preferred embodiment, the method further comprises a step id) of determining the expression profile of genes linked to the immune system, in particular cytokines, within the skin explant and whereby the step ii) also makes it possible to determine the inflammatory potential associated with this same composition.

According to a final preferred embodiment, the method further comprises a step ie) of determining the degree of mast cells degranulation within the skin explant and whereby step ii) also makes it possible to determine the inflammatory potential associated with this same composition.

### BRIEF DESCRIPTION OF THE DRAWING

FIG. 1 represents the different populations of antigen-presenting cells (APC) and the markers associated with each of them.

### DETAILED DESCRIPTION OF THE INVENTION

By “vaccine composition”, it is meant a composition comprising at least one antigen (lipid, carbohydrate, protein or even peptide) or a nucleic acid encoding at least one antigen and, potentially, at least one adjuvant.

By “skin explant”, it is meant a fragment of skin which comprises a thickness of at least 5 mm of hypodermis (preferably between 5 and 15 mm of hypodermis and, preferably still, between 5 and 10 mm of hypodermis) in addition to the epidermis, to the dermis and to the skin appendages.

The skin appendages correspond to hair follicles, sebaceous glands and sweat glands. The hypodermis is the layer of tissue that is located immediately below the dermis of the skin. The hypodermis is a loose connective tissue that is well-vascularized and also contains adipose tissue and immune cells.

If this skin explant is taken from a mammal, one may opt for human or pig. Now, given the preferred usage of the method according to the invention, one would rather opt for a human skin explant.

In connection with the origin of the skin explant, it can be obtained from a surgical procedure originating in any part of the body, including surgeries of the abdomen, chest, buttocks, back, or even, why not, the scalp or any other part of the body comprising skin. In the case of a cylindrical skin explant, one would therefore opt more for a skin explant with a diameter of between 10 mm and 50 mm, preferably between 15 mm and 40 mm.

Ideally, the skin explant would be positioned within an insert that can take multiple forms and, in particular, corresponds to a suspended or standing insert. Currently, a suspended insert is preferable. The bottom of this insert comprises a porous membrane. Typically, this porous membrane has a diameter between 5 mm and 40 mm, preferably between 9.5 mm and 30 mm. This porous membrane will have a porosity between 0.4 μm and 8 μm, preferably between 0.4 μm and 1.5 μm, with 0.8 μm to 1.2 μm as the most preferred porosity. In terms of material, one can therefore choose a porous membrane from polyethylene terephthalate (PET), nitrocellulose, and polycarbonate membranes. Finally, and as an example of such inserts, one could cite those supplied by the companies NUNC, CORNING, BECTON DICKINSON (BD FALCON), MILLIPORE (MILLICELL) which may take the form of inserts with a polycarbonate, PET, or nitrocellulose membrane, and which are prepackaged in multi-well plates of 6, 8, 12, or 24-well culture plates, and whose membrane porosity can vary from 0.4 μm to 8 μm.

The epidermis is in contact with the atmosphere, whereas the dermis, the epidermal appendages and the thickness of at least 5 millimeters of hypodermis are immersed in the culture medium.

The skin explant may be prepared as described in international application WO 2019/170281. In detail, said skin explant was submerged, with the exception of the epidermis, in a liquid matrix capable of solidifying like blood plasma, a solution derived from blood plasma (e.g., a dilution of blood plasma in physiological buffer, in particular diluting blood plasma to at least 10%, 20%, 30%, or even at least 40% by weight (weight/matrix total weight)), a fibrinogen solution, a collagen solution, a gelatin solution, synthetic polymer solutions, natural polymer solutions (e.g. agarose (low melting/low melting point agarose or agar), starch, polysaccharides), and mixtures thereof. For more details relating to the matrices capable of solidifying and the methods for placing the skin explants therein, one can consult European patent No. EP 2 88 2 290 B1. Now, these matrices and methods described in paragraphs [0024] to [0042] and [0067] to [0080] of European Patent No. EP 2 882 290 B1 are incorporated into this current patent application by reference.

Now, the skin explant may be also prepared as disclosed in US patent application US 2019/0219564. Accordingly, the cell culture insert is a skin sample holder applying tension to the skin biopsy. In detail, the skin sample holder comprises a base frame, with a skin biopsy receiving surface upon which at least part skin biopsy may be placed and which extends across an area defined by the shape of the frame; and a securing member which is releasably connectable to the base frame and a grip which holds the skin biopsy under tension. Finally, the bottom of the base frame comprises a porous membrane on which the hypodermis rests. Considering the skin tension, it is typically comprised in the range of 0.22 to 0.57N, and preferably in the range of 0.29 to 0.46N.

By “transcutaneous administration”, it is meant the administration of a composition that enables it to penetrate the skin barrier. Such a transcutaneous administration can be accomplished subcutaneously (that is to say via a subcutaneous injection (a needle), transdermally (via a simple patch or with microneedles) or topically.

By “topical administration”, it is meant an application of the composition to be tested to the epidermis of the skin explant in the form of a cream or a gel for example.

By “subcutaneous administration”, it is meant a subcutaneous injection, which is therefore carried out in the hypodermis of the skin explant using a needle, which is also why it is called a “hypodermic” injection. This type of injection, which is well known to those skilled in the art, generally requires making a skin fold using the fingers and the subcutaneous injection is then performed in the skin fold.

By “transdermal administration”, which is also “transepidermal administration”, it is meant an administration which uses a patch integrating, or not, microneedles (said microneedles can be biodegradable).

According to a preferred embodiment, step ia) consists of a subcutaneous or transdermal administration of a composition comprising the substance in a skin explant.

The composition in question is a composition to be tested which is in liquid form. Ideally, the volume of this composition is between 10 μl and 1 ml, preferably between 10 μl and 500 μl and, particularly preferably, between 10 μl and 200 μl.

The needle for injecting the composition typically has sufficient length to reach the hypodermis. Therefore, it is preferable to utilize needles with a length greater than or equal to 10 mm. By way of example for such needles, needles can be used with a length of 12, 16, 20, 25, 30, 35, 40 or even 45 mm. Ideally, therefore, the needle has a length of between 16 and 45 mm, preferably a length of between 20 and 40 mm. As for the diameter of the needle to be used, it can be identified simply by those skilled in the art based on their general knowledge. Typically, such hypodermic needles are of the gauges 18G, 19G, 20G, 21G, 22G, 23G, 25G, 26G, 27G, 28G, 29G, 30G or even 31G.

This injection step can be carried out by an experimenter, who pinches to allow the formation of a skin fold and thus facilitate the subcutaneous injection. Now, this injection step can also be performed by an automatic injection device. Typically, the device allows an injection at a determined depth, relative to the surface of the epidermis, so as to obtain a subcutaneous injection.

Ideally, only one step ia) of the administration of a composition is carried out per skin explant.

The step ib) of determining the activation status of immune cells and, in particular, of antigen-presenting cells within the skin explant, is carried out by monitoring the expression of activation markers within these cells.

To do this, one may use the well-known methods of immunohistochemistry that utilize the fixation of the skin explant, the embedding of the explant (e.g., paraffin, OCT, EPON), before its conservation, and finally the production of histological sections from the embedded block. The details of such methods are described for example in “Immunohistochemistry: Basics and Methods” by Igor BUCHWALOW (SPRINGER Editions).

Methods such as flow cytometry and BULK and SINGLE-CELL types transcriptomic analyses can also be used.

The histological sections that can be used may have a very significant thickness of up to 500 μm. Ideally, the histological section will thus have a thickness of between 1 and 500 μm. Currently, it is possible to use sections with more classic dimensions with a thickness between 2 and 25 μm.

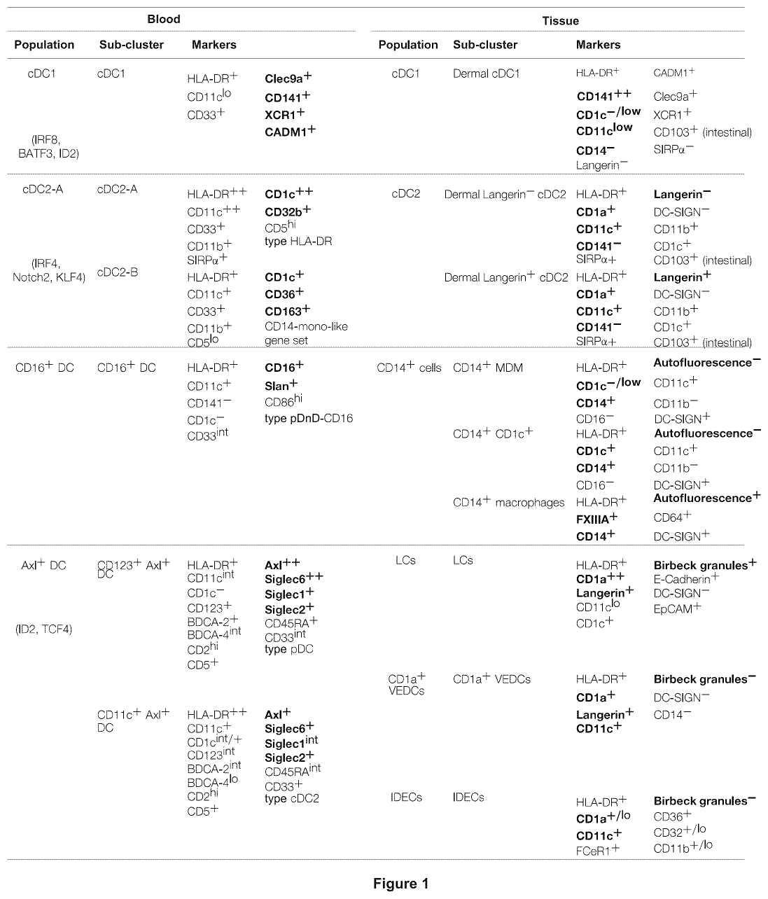

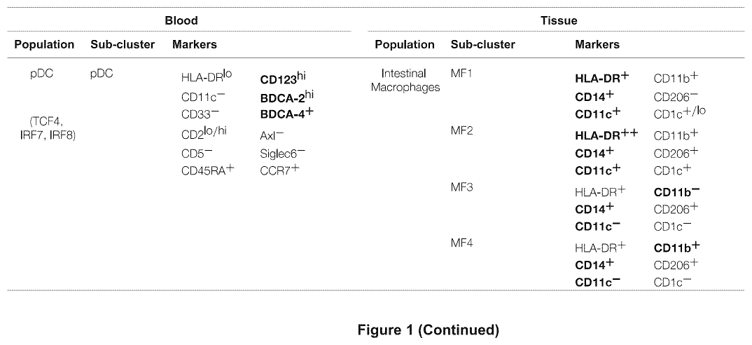

Antigen-presenting cells are defined as cells expressing the CD45 and HLA-DR markers. For more detail, the sets of antigen-presenting cells are described in FIG. 1.

Advantageously, the antigen-presenting cells (APC) are selected from dermal cDC1 cells, dermal langerin⁻ cDC2 cells, dermal langerin⁺ cDC2 cells and Langerhans cells (LCs). The details of the markers profile expressed by these cells are given in Table 1 below.

TABLE 1

| Cells | Markers |
|---|---|
| Dermal cDC1 | HLA-DR⁺, CD141⁺⁺, CD1c^{-/weak}, |
| | CD11c^{weak}, CD14⁻, langerin⁻, CADM1⁺, |
| | CLEC9A⁺, XCR1⁺, SIRPα⁻ |
| Dermal langerin⁻ cDC2 | HLA-DR⁺, CD1a⁺, CD11c⁺, |
| | CD141⁻, SIRPα⁺, langerin⁻, |
| | DC-SIGN⁻, CD11b⁺, CD1c⁺ |
| Dermal langerin⁺ cDC2 | HLA-DR⁺, CD1a⁺, CD11c⁺, |
| | CD141⁻, SIRPα⁺, langerin⁺, |
| | DC-SIGN⁻, CD11b⁺, CD1c⁺ |
| Langerhans cells (LCs) | HLA-DR⁺, CD1a⁺⁺, langerin⁺, |
| | CD11c^{weak}, CD1c⁺, Birbeck⁺ granules, |
| | E-cadherin⁺, DC-SIGN⁻, EpCAM⁺ |

By “activation markers”, it is meant all the markers described in Table 2.

TABLE 2
Activation markers of antigen-presenting cells

CD40
CCR7
CD86
CD83
CD80
HLA-DR

An antigen-presenting cell is considered activated when at least 2 activation markers are overexpressed in this cell, preferably at least 3 activation markers and, particularly preferably, at least 5 activation markers.

By “overexpression of an activation marker”, it is meant an increase in its expression of at least 20%, preferably an increase of at least 30% compared to the basal level of expression. Such an increase is measured between the antigen-presenting cells of two skin explants from the same donor, only, one of which was injected with the composition whose vaccine potential is to be determined and the other of which was injected with a control composition (water for injection or PBS).

Step ic) of determining the possible migration of antigen-presenting cells within the skin explant is also carried out by well-known immunohistochemistry methods as described previously.

To do this, one determines the relocation or not of the antigen-presenting cells near or not to the lymphatic and/or blood vessels. The determination of such a relocation can be achieved simply to the extent that the blood and lymphatic vessels exhibit characteristic structures that are easily identifiable within the histological section of the skin explant.

By “relocation of antigen-presenting cells near or not to blood and/or lymphatic vessels”, it is meant an increase of at least 10% in the population of antigen-presenting cells near to blood and lymphatic vessels, preferably of at least 20%.

Step ii) of determining the vaccine potential of the composition can then be simply carried out with regard to the result of steps ib) and, possibly ic).

This will make it possible to determine whether the composition has vaccine potential in terms of increased activation of antigen-presenting cells.

Thus, a composition associated with an activation of at most 10% of antigen-presenting cells have low or even zero vaccine potential.

Conversely, a composition associated with activation of at least 40% of antigen-presenting cells exhibits a high vaccine potential.

Preferably, an increase of at least 20% on at least two of the activation markers (HLA-DR, CD80, CD86, CD83, CD40 and CCR7) is observed in the antigen-presenting cells in the case of a composition associated with a high vaccine potential.

Finally, a composition associated with an intermediate activation of antigen-presenting cells and not exceeding either of the two previous thresholds, exhibits an intermediate, or moderate, vaccine potential.

Also, it is possible to specify the vaccine potential of the composition with regard to possible migration of antigen-presenting cells.

Thus, a composition associated with a relocation of at most 2% of antigen-presenting cells near blood and/or lymphatic vessels exhibits low, or even zero, vaccine potential.

Conversely, a composition associated with a relocation of at least 10% of antigen-presenting cells near blood and/or lymphatic vessels exhibits a high vaccine potential.

For control purposes, the same steps ia), ib), ic) may be carried out with a negative control (e.g. PBS) and/or with a composition comprising a positive control corresponding to a composition known to possess high vaccine potential.

According to a preferred embodiment, the method further comprises a step id) determination of the expression profiles of genes linked to the immune system within the skin explant.

By way of example of genes related to the immune system, one can cites, in a non-limiting manner, the following genes:

ABCB1, ABCF1, ABL1, ACKR4, ADA, ADGRE5, AHR, AICDA, AIRE, ALAS1, APP, ARG1, ARG2, ARHGDIB, ATG10, ATG12, ATG16L1, ATG5, ATG7, ATM, B2M, B3GAT1, BATF, BATF3, BAX, BCAP31, BCL10, BCL2, BCL2L11, BCL3, BCL6, BID, BLNK, BST1, BST2, BTK, BTLA, C1QA, C1QB, C1QBP, C1R, C1S, C2, C3, C4A/B, C4BPA, C5, C6, C7, C8A, C8B, C8G, C9, CAMP, CARD9, CASP1, CASP10, CASP2, CASP3, CASP8, CCL11, CCL13, CCL15, CCL16, CCL18, CCL19, CCL2, CCL20, CCL22, CCL23, CCL24, CCL26, CCL3, CCL4, CCL5, CCL7, CCL8, CCND3, CCR1, CCR10, CCR2, CCR5, CCR6, CCR7, CCR8, CCRL2, CD14, CD160, CD163, CD164, CD19, CD1A, CD1D, CD2, CD209, CD22, CD24, CD244, CD247, CD27, CD274, CD276, CD28, CD34, CD36, CD3D, CD3E, CD3EAP, CD4, CD40, CD40LG, CD44, CD45R0, CD45RA, CD45RB, CD46, CD48, CD5, CD53, CD55, CD58, CD59, CD6, CD7, CD70, CD74, CD79A, CD79B, CD80, CD81, CD82, CD83, CD86, CD8A, CD8B, CD9, CD96, CD99, CDH5, CDKN1A, CEACAM1, CEACAM6, CEACAM8, CEBPB, CFB, CFD, CFH, CFI, CFP, CHUK, CIITA, CISH, CLEC4A, CLEC4E, CLEC5A, CLEC6A, CLEC7A, CLU, CMKLR1, CR1, CR2, CRADD, CSF1, CSF1R, CSF2, CSF2RB, CSF3R, CTLA-4, CTLA4-TM, CTNNB1, CTSC, CTSG, CTSS, CUL9, CX3CL1, CX3CR1, CXCL1, CXCL10, CXCL11, CXCL12, CXCL13, CXCL2, CXCL8, CXCL9, CXCR1, CXCR2, CXCR3, CXCR4, CXCR6, CYBB, DEFB1, DEFB103A, DEFB103B, DEFB4A, DPP4, DUSP4, EBI3, EDNRB, EEF1G, EGR1, EGR2, ELP1, ENTPD1, EOMES, ETS1, FADD, FAS, FCAR, FCER1A, FCER1G, FCGR1A/B, FCGR2A, FCGR2A/C, FCGR2B, FCGR3A/B, FCGRT, FKBP5, FN1, FOXP3, FYN, G6PD, GAPDH, GATA3, GBP1, GBP5, GFI1, GNLY, GP1BB, GPI, GPR183, GUSB, GZMA, GZMB, GZMK, HAMP, HAVCR2, HFE, HLA-A, HLA-B, HLA-C, HLA-DMA, HLA-DMB, HLA-DOB, HLA-DPA1, HLA-DPB1, HLA-DQA1, HLA-DQB1, HLA-DRA, HLA-DRB1, HLA-DRB3, HPRT1, HRAS, ICAM1, ICAM2, ICAM3, ICAM4, ICAM5, ICOS, ICOSLG, IDO1, IFI16, IFI35, IFIH1, IFIT2, IFITM1, IFNA1/13, IFNA2, IFNAR1, IFNAR2, IFNB1, IFNG, IFNGR1, IFNL1, IFNL2, IFNL2/3, IGF2R, IKBKB, IKBKE, IKBKG, IKZF1, IKZF2, IKZF3, IL10, IL10RA, IL11RA, IL12A, IL12B, IL12RB1, IL13, IL13RA1, IL15, IL16, IL17A, IL17B, IL17F, IL18, IL18R1, IL18RAP, IL19, IL1A, IL1B, IL1R1, IL1R2, IL1RAP, IL1RL1, IL1RL2, IL1RN, IL2, IL20, IL21, IL21R, IL22, IL22RA2, IL23A, IL23R, IL26, IL27, IL2RA, IL2RB, IL2RG, IL3, IL32, IL4, IL4R, IL5, IL6, IL6R, IL6ST, IL7, IL7R, IL9, ILF3, IRAK1, IRAK2, IRAK3, IRAK4, IRF1, IRF3, IRF4, IRF5, IRF7, IRF8, IRGM, ITGA2B, ITGA4, ITGA5, ITGA6, ITGAE, ITGAL, ITGAM, ITGAX, ITGB1, ITGB2, ITLN1, ITLN2, JAK1, JAK2, JAK3, KCNJ2, KIR3DL1, KIR3DL2, KIR3DL3, KIR_Activating_Subgroup_1, KIR_Activating_Subgroup_2, KIR_Inhibiting_Subgroup_1, KIR_Inhibiting_Subgroup_2, KIT, KLRA1P, KLRB1, KLRC1, KLRC2, KLRC3, KLRC4, KLRD1, KLRF1, KLRF2, KLRG1, KLRG2, KLRK1, LAG3, LAIR1, LAMP3, LCK, LCP2, LEF1, LGALS3, LIF, LILRA1, LILRA2, LILRA3, LILRA4, LILRA5, LILRA6, LILRB1, LILRB2, LILRB3, LILRB4, LILRB5, LITAF, LTA, LTB4R, LTB4R2, LTBR, LTF, LY96, MAF, MALT1, MAP4K1, MAP4K2, MAP4K4, MAPK1, MAPK11, MAPK14, MAPKAPK2, MARCO, MASP1, MASP2, MBL2, MBP, MCL1, MIF, MME, MR1, MRC1, MS4A1, MSR1, MUC1, MX1, MYD88, NCAM1, NCF4, NCR1, NFATC1, NFATC2, NFATC3, NFIL3, NFKB1, NFKB2, NFKBIA, NFKBIZ, NLRP3, NOD1, NOD2, NOS2, NOTCH1, NOTCH2, NT5E, OAZ1, PAX5, PDCD1, PDCD1LG2, PDCD2, PDGFB, PDGFRB, PECAM1, PIGR, PLA2G2A, PLA2G2E, PLAAT4, PLAU, PLAUR, PML, POLR1B, POLR2A, POU2F2, PPARG, PPBP, PPIA, PRDM1, PRF1, PRKCD, PSMB10, PSMB5, PSMB7, PSMB8, PSMB9, PSMC2, PSMD7, PTAFR, PTGER4, PTGS2, PTK2, PTPN2, PTPN22, PTPN6, PTPRC, PYCARD, RAF1, RAG1, RAG2, RELA, RELB, RORC, RPL19, RTRAF, RUNX1, S100A8, S100A9, S1PR1, SDHA, SELE, SELL, SELPLG, SERPING1, SH2D1A, SIGIRR, SKI, SLAMF1, SLAMF6, SLAMF7, SLC2A1, SMAD3, SMAD5, SOCS1, SOCS3, SPP1, SRC, STAT1, STAT2, STAT3, STAT4, STAT5A, STAT5B, STAT6, STING1, SYK, TAGAP, TAL1, TAP1, TAP2, TAPBP, TBK1, TBP, TBX21, TCF4, TCF7, TFRC, TGFB1, TGFBI, TGFBR1, TGFBR2, THY1, TICAM1, TIGIT, TIRAP, TLR1, TLR2, TLR3, TLR4, TLR5, TLR7, TLR8, TLR9, TNF, TNFAIP3, TNFAIP6, TNFRSF10C, TNFRSF11A, TNFRSF13B, TNFRSF13C, TNFRSF14, TNFRSF17, TNFRSF1B, TNFRSF4, TNFRSF8, TNFRSF9, TNFSF10, TNFSF11, TNFSF12, TNFSF13B, TNFSF15, TNFSF4, TNFSF8, TOLLIP, TP53, TRAF1, TRAF2, TRAF3, TRAF4, TRAF5, TRAF6, TUBB, TYK2, UBE2L3, VCAM1, VTN, XBP1, XCL1, XCR1, ZAP70, ZBTB16, ZEB1 and sCTLA4.

Step ii) of determining the vaccine potential of the composition thus enables, in conjunction with said step id), to specify the vaccine potential of the composition.

Preferably, the genes related to the immune system are cytokines.

Step ii) of determining the vaccine potential of the composition thus enables, in conjunction with step id), to determine the inflammatory potential associated with this same composition.

By way of example of usable cytokines, one can cite interleukins and their receptors. By way of example of interleukins, one can cite IL-1A, IL-1B, IL-5, IL-6, IL-7, IL-8, IL-9, IL-10, IL-12, IL-17A, IL-17C, IL-17F, IL-19, IL-21, IL-22, IL-23, IL-27, IL-31 and IL-33, and as examples of interleukin receptors, one can cite IL-10RA, IL-10RB, IL-1R1, IL-5RA (CD125) and IL-9R.

As examples of usable cytokines, one can also cite chemokines, which are chemotactic cytokines that control not only the migration patterns and positioning of immune cells, and also their receptors. By way of example of cytokines, one can cite C5, Eotaxin, MCP-4, TARC, MCP-1, MIP-3A, CCL22, CCL23, MIP-1B, RANTES, MCP-3, MCP-2, CX3CL1, IL8RA, INP10, L8RB and CXCL3. By way of example of chemokine receptors, one can cite CCL13 (MCP-4), CCR1, CCR2, CCR3, CCR4, CCR5, CCR6, CCR8, CX3CR1, CXCR1 and CXCR2.

It should be noted that other cytokines (than interleukins and chemokines) can be envisaged as markers of inflammation. As examples of such cytokines, one can cite MCP-1, GM-CSF, TNFSF5, M-CSF, G-CSF, TNFSF6, IFNA2, IFNG, TNFA, TNFB, MIF, NAMPT, TRAIL et IFNA1.

Ideally, the cytokines are chosen from GM-CSF, MIP-1a, MCP-1, IL-2, IL-4, IL-6, IL-8, IL-13, IL-12, IL-15, IL-16, MIP-3a, IP-10, MIP-1a, MIP-1b, MDC, IL-27, and Eotaxin-3.

The expression profile of cytokines can be determined within the skin explant, the culture medium or even within the matrix.

Now, the inventors have shown that the expression profile of cytokines within the matrix shows both greater diversity and higher expression levels.

Ideally, therefore, the determination of the cytokine expression profile is performed within the matrix of the skin explant.

According to a preferred embodiment, the method further comprises a step id) determining the level of degranulation of mast cells within the skin explant.

Step ii) of determining the vaccine potential of the composition will thus make it possible, in conjunction with step ie), to again determine the inflammatory potential associated with this same composition.

Step ie) of determining the level of mast cell degranulation can be carried out using techniques well known to those skilled in the art. Techniques that can be used for this step include ELISA or colorimetric techniques to measure the presence of inflammatory mediators contained in mast cell granules, such as histamine or tryptase, or secreted de novo such as lipid mediators or cytokines/chemokines, or even fluorescence, immunofluorescence, or fluorochrome techniques specific to mast cell granules.

Ideally, this step ie) is carried out within a maximum of 6 hours following the administration step ie), preferably within a maximum of 4 hours.

According to a preferred embodiment, step ie) of determining the level of degranulation of mast cells within the skin explant is carried out by fluorescence analysis.

Ideally, this step ie) of determining the level of mast cell degranulation uses avidin.

Avidin is in fact a glycoprotein which binds very specifically to the heparin contained in the granules of mast cells (THARP et al., J. Histochem. Cytochem., vol.33, p: 27-32, 1985). Thus, during the degranulation process once the granules are externalized they become directly accessible to avidin. Now, and in the case of tissue fixation, the intracellular granules become accessible to avidin as soon as tissue permeabilization is carried out. This avidin can be conjugated to a fluorochrome (avidin-FITC, avidin-Alexa Fluor™ 488, avidin-sulforhodamine 101 or any other fluorescent molecule) or with a bioluminescent molecule. Now, it is also possible to use avidin alone (unconjugated) and in combination with a molecule that is complementary to it. The following examples of such a molecule may be cited: biotin conjugated to a fluorochrome, a bioluminescent molecule, or any other identifiable molecule.

Currently, the determination of the extent of mast cell degranulation can use other markers, in particular the nucleus or the plasma membrane, so as to facilitate the identification of granules externalized from mast cells It will also be possible to measure in the culture medium tryptase, histamine, beta-hexosaminidase, chymase or any other molecules preformed within the mast cell granules and released during degranulation.

Typically, the determination of the level of mast cell degranulation within the skin explant can be accomplished by following the protocol described in GAUDENZIO et al. (J. Clin. Invest., vol.126, p: 3981-3998, 2016)

Ideally, step ie) of determining the level of mast cell degranulation will be performed on at least one histological section taken from the skin explant.

To do this, one may use the well-known methods of immunohistochemistry that utilize the fixation of the skin explant, the embedding of the explant (e.g., paraffin, OCT, EPON), before its conservation, and finally the production of histological sections from the embedded block. The details of such methods are described for example in Immunohistochemistry: Basics and Methods by Igor BUCHWALOW (Editions SPRINGER).

The usable histological sections may have a very significant thickness of up to 500 μm. Ideally, the histological section will thus have a thickness of between 1 μm and 500 μm. Though, it is possible to use sections with more classic dimensions with a thickness between 2 and 25 μm.

As for determining the level of mast cell granulation within the explant itself, one first determines, for each identified mast cell, whether it is associated with low, moderate, or high degranulation, then determines the proportion (percentage) of mast cells associated with each of these levels of degranulation (low, moderate, and high). The level of degranulation can also be analyzed in an automated manner via image analysis software, computer algorithm, or artificial intelligence techniques such as “machine-learning” or “deep-learning”.

In detail, a mast cell with a low level of degranulation corresponds to a mast cell with 0 to 2 granules around it (or to a cell with a smooth outline); a mast cell with a moderate level of degranulation corresponds to a mast cell with 3 to 6 granules around it (or to a cell with a granular appearance); and a mast cell with a high level of degranulation corresponds to a mast cell with more than 6 granules around it (or to a cell with a distorted shape).

It will therefore be possible to determine whether the composition presents an inflammatory potential with regard to the percentage of mast cell degranulation as determined at the end of step ie).

Thus, a substance associated with a proportion of more than 50% of granulocytes presenting a low level of degranulation and/or of less than 10% a high level of degranulation has a low, or even zero, inflammatory potential.

Conversely, a substance associated with a proportion of granulocytes of more than 50% presenting a high level of degranulation indicates a high inflammatory potential.

The following examples are given solely for the purpose of illustrating the subject matter of the present invention, of which they in no way constitute a limitation.

### EXAMPLES

### 1—Preparation of Skin Explants

Skin explants are prepared from complete skin samples from different donors and include the epidermis, dermis, and hypodermis (1.5 cm to 2 cm). The explants (epidermis, dermis, and hypodermis) are then cut using a circular metal punch to obtain cylinders of 11 mm to 20 mm in diameter, where the thickness of the hypodermis is adjusted to the desired value (0.5 cm to 1 cm). Finally, these explants were kept floating in a buffered saline solution until the “inclusion” step in the solidified matrix. This inclusion step was done with a process similar to that used for the NATIVESKIN™ model. Briefly, the skin explant is delicately placed into an insert (8-well MILLICELL™ chamber slide) with a porous membrane at the bottom (in PET, porosity 1 μm) and containing a blood plasma-derived solution that has been treated with an anticoagulant that undergoes reversal in the presence of calcium ions (sodium citrate). This solution consists of 42% of blood plasma, 50% of 0.9% NaCl solution, 8% of 1% CaCl2 saline solution, an antifibrinolytic agent (tranexamic acid or aprotinin), and low melting point agarose at 0.7% (LMP Agarose, GIBCOBRL, LIFE TECHNOLOGIES) (melted in an incubator at 65.5° C.). The antifibrinolytic agent functions to inhibit enzymes that can degrade the plasma matrix, these enzymes being secreted by the skin explant, and thereby maintains the integrity of the explant.

### 2—Antigen Presenting Cells and Skin Explant

### 2.1—Identification of Antigen-Presenting Cells Within the Skin Explant

The presence of antigen-presenting cells within the explant was analyzed during culture and in the days following using several techniques immunohistochemistry coupled with artificial intelligence (multiplexed imaging), single-cell transcriptomic analysis and flow cytometry. Thus, single-cell transcriptomic analysis highlighted the presence, in the model and compared to human skin, of comparable proportions of immune cells (expressing PTPRC), stromal cells (expressing VIM), melanocytes (expressing MLANA), keratinocytes (expressing KRT14 and KRT1) and adipocytes over a period of 10 days. Possible changes in the transcriptome of all these cells were investigated over time. Of the 2,000 genes detected in the experiment, only a small variation of only 55 genes in the immune compartment was detected between day 0 and day 5 of culture with a return to normal between day 5 and day 10 of culture. These data indicate that structural and immune cells contained in HYPOSKIN models remain phenotypically and functionally stable for a period of at least 10 days.

In conclusion, the results unexpectedly showed that antigen-presenting cells within the skin explant exhibit similar numbers and diversity to those observed within the skin in vivo. Moreover, the results also showed that this number and diversity of presenting cells is maintained from the first to the tenth day of culture.

### 2.2—Demonstration of Maturation of Antigen-Presenting Cells Within the Skin Explant

In view of the previous result, the inventors questioned the maturation capacity of the antigen-presenting cells within the skin explant.

Also, and to test this maturation capacity, several techniques were used: immunohistochemistry coupled with artificial intelligence (multiplexed imaging), single-cell transcriptomic analysis and flow cytometry.

Again, and unexpectedly, the results showed that in addition to preserving their number and diversity, antigen-presenting cells also exhibit an intact maturation capacity.

Therefore, the skin explant can be used to test the ability of an antigenic composition to activate antigen-presenting cells. At the same time, and as previously demonstrated by the inventors, it is possible to test the inflammatory potential associated with this same composition.

### 3—Confirmation of the vaccine potential of the composition INFLUVAC TETRA

125 μL of INFLUVAC TERTR vaccine solution was injected into the adipose tissue of skin explants from 3 separate donors using a syringe and a 27G needle of 12 mm in length. As a negative control, 100 μL of water was injected into the adipose tissue of skin explants from the same donors.

The explants were then cultured (incubator at 37° C., 5% CO2 and water-saturated air) for 8 h, 24 h and 48 h.

A staining of hematoxylin and eosin was performed on skin explants from each donor before injection and 48 hours after the injection of water or the vaccine composition. At the same time, the presence of possible DNA fragmentation was tested in these same skin explants.

The results showed no alteration of cellular integrity 48 hours after injection of the vaccine composition for the 3 donors.

After evaluating the stability and viability of the model during the study, the production of cytokines by the models was studied. In order to obtain optimal results, the assay was performed on the culture medium, matrix and lysate of the models injected with water and vaccine at 8 hours of culture for the three donors of the study.

One can observe numerous unspecified values of cytokine concentrations for the lysate, because their concentration is below the detection limit of the MSD device. Regarding the assay for the culture medium, it allows the concentration of most of the cytokines in the panel to be measured, but in a quantity significantly lower than the assay for the matrix, even if the concentration trends are the same for these two assay media. Therefore, the matrix will be used for future cytokine assay analyses, as it allows the detection of the majority of cytokines in sufficient concentrations.

Following optimization of the MSD assay, the concentrations of 36 cytokines were measured in the matrix of the study models. The uninjected models appear to be the least inflamed and have the lowest overall cytokine concentrations. At 8 hours after injection, a slight increase in cytokine concentration was observed. A clear increase in cytokine production was observed in the 3 donors tested 24 hours after injection with higher cytokine concentrations in the vaccinated models, notably IFN-g, TNF-a, IL-1a, IP-10, eotaxin-3, IL-12p40, IL-13, IL-15, IL-16, MCP-4, MIP-1a, MIP-1b, MIP-3a, IL-1b, GM-CSF, MCP-1 and TARC.

Finally, the cytokine concentration increases slightly 48 hours after injection, particularly in the vaccinated models. Overall, one therefore observes a biological response following vaccination via the production of cytokines and chemokines linked to the activation of the immune system in the models over time, in particular with a peak 24 hours after the injection.

To determine the presence of antigen-presenting cells, skin explants from each donor before injection, 8 h, 24 h and 48 h after the injection of water or vaccine composition were cryopreserved at −80° C. for immunohistochemical analysis or embedded in paraffin for analysis of mast cell degranulation.

Cryopreserved sections were then labeled with different antibodies specific to antigen-presenting cells (anti CD45, CD207, CD1c, CD40, CCR7, CD80, CD86, CD83, and HLA-DR).

Analysis of these sections showed that injection of the vaccine composition leads to the activation of antigen-presenting cells (APC) within the explant, as well as the migration of a certain number of them. Therefore, the results show that the administered vaccine is associated with the establishment of adaptive immunity.

Paraffin sections were used at the same time to determine the level of mast cell degranulation within the tissue under the different conditions. To do this, one performs labeling using avidin coupled with a fluorochrome which allows for the detection of mast cell granules. The first step involves deparaffinizing and rehydrating the sections fixed and embedded in paraffin. The sections are incubated for 30 minutes at room temperature in Citrate pH6 buffer, then saturated and permeabilized for 40 minutes at 37° C. with a solution of goat serum and 0.1% Triton. The sections are then incubated for one hour at room temperature in a humid chamber with 5 μg/mL Avidin-Sulforhodamine 101 (Avidin TEXAS RED, MERCK). Staining of the cell nuclei is then carried out by incubating the sections with DAPI (D9542, SIGMA) at 1/1000 for 3 minutes at room temperature. The mounting medium is added and a coverslip is placed on the sections. The slides are then analyzed under a fluorescence microscope to determine the level of mast cell degranulation in the different explants.

The results of these degranulation experiments are presented in Table 3.

TABLE 3

| Percentage of | | |
|---|---|---|
| granulocytes | | Vaccine |
| with | Water | composition |
| Low level of | 15% | 15% |
| degranulation | | |
| Moderate level of | 30% | 30% |
| degranulation | | |
| High level of | 60% | 60% |
| degranulation | | |
| Inflammatory | 30% | 30% |
| potential | | |

The results made it possible to determine the level of degranulation of mast cells identified in the samples and, after integration, to deduce the inflammatory potential of the injected composition. In this case, the results show that the administered vaccine is not associated with an inflammatory risk due to mast cell degranulation.

### 4—Confirmation of the Vaccine Potential of the Composition INFLUVAC TETRA

The analysis of the INFLUVAC TETRA vaccine composition made it possible to demonstrate that this vaccine induces the expression of a cytokine signature which indicates the activation of the cutaneous immune system, in particular the expression of GM-CSF, MIP-1a, MCP-1, IL-2, IL-4, IL-6, IL-8, IL-13, IL-12, IL-15, IL-16, MIP-3a, IP-10, MIP-1a, MIP-1b, MDC, IL-27 and Eotaxin-3 between 8 h and 24 h of culture. Using multiplexed imaging technique, it was demonstrated that the vaccine induces the activation of skin dendritic cells and Langerhans cells via an increase in the expression of the maturation/activation markers CD40, CCR7, CD80, CD86, CD83 and HLA-DR.

1. An in vitro method for determining the vaccine potential of a composition comprising the steps of:
ia) transcutaneous administration of the composition to a skin explant, comprising the epidermis, dermis and skin appendages as well as a thickness of at least 5 millimeters of hypodermis;
ib) determination of the activation status of antigen-presenting cells within the skin explant; and
ii) determination of the vaccine potential of the composition.

2. The method of claim 1, wherein the antigen-presenting cells are chosen from dermal cDC1 cells, dermal langerin⁻ cDC2 cells, dermal langerin⁺ cDC2 cells and Langerhans cells.

3. The method of claim 1, wherein the step ia) consists of a subcutaneous or transdermal administration of a composition comprising the substance in a skin explant.

4. The method of claim 1, wherein an antigen-presenting cell is considered activated when at least 2 activation markers are overexpressed in this cell, said activation markers being selected from the group consisting of CD40, CCR7, CD86, CD83, CD80, and HLA-DR.

5. The method of claim 4, wherein the overexpression of an activation marker corresponds to an increase in its expression of at least 20%.

6. The method of claim 1, wherein the step ii) of determining the vaccine potential of the composition is carried out with regard to the proportion of antigen-presenting cells activated at the end of step ia), with:
a composition associated with an activation of at most 10% of antigen-presenting cells exhibits low or even zero vaccine potential; and
a composition associated with activation of at least 40% of antigen-presenting cells exhibits a high vaccine potential.

7. The method of claim 1, wherein said method further comprises a step ic) of determining the possible migration of the antigen-presenting cells within the skin explant.

8. The method of claim 1, wherein said method further comprises a step id) of determining the expression profile of the cytokines within the skin explant and in that step ii) also makes it possible to determine the inflammatory potential associated with this same composition.

9. The method of claim 8, wherein the determination of the cytokine expression profile is performed within the matrix of the skin explant.

10. The method of claim 1, wherein said method further comprises a step ie) of determining the level of degranulation of mast cells within the skin explant and in that step ii) also makes it possible to determine the inflammatory potential associated with this same composition.

11. The method of claim 1, wherein the skin explant is positioned within an insert.

12. The method of claim 11, wherein the insert is a suspended or a standing insert.

13. The method of claim 11, wherein the bottom of the insert comprises a porous membrane.
